(19) United States
(12) Patent Application Publication
(10) Pub. No.: US 2018/0213789 A1
(43) Pub. Date: Aug. 2, 2018

(54) ANTIMICROBIAL COMPOSITIONS FOR SURGICAL APPLICATIONS

(71) Applicant: TELEFLEX MEDICAL INCORPORATED, Morrisville, NC (US)

(72) Inventors: NISHA GUPTA, READING, PA (US); KAMNA GIARE-PATEL, READING, PA (US); CHUANTING YOU, READING, PA (US)

(21) Appl. No.: 15/746,996

(22) PCT Filed: Jul. 22, 2016
(86) PCT No.: PCT/US2016/043543

§ 371 Date: Jan. 23, 2018

Related U.S. Application Data
(60) Provisional application No. 62/196,424, filed on Jul. 24, 2015.

(51) Int. Cl. A01N 47/44; A61K 31/155; A61P 31/04; A61K 47/10; A61K 47/34; A61L 2/18; A61L 2/00; B05D 3/02; B05D 1/18

(52) U.S. Cl. CPC A01N 47/44; A61K 31/155; A61P 31/04; A61K 47/10; A61L 2202/24; A61L 2/18; A61L 2/0088; B05D 3/0254; B05D 1/18; A61K 47/34

(57) ABSTRACT

An antimicrobial composition comprising alexidine, a solvent, an optional carrier polymer and one or more excipient or additives is disclosed. The antimicrobial composition is used to provide antimicrobial properties to a surgical device during surgery and after surgery. The antimicrobial composition is also used to disinfect a surgical device or a surgical site.

### TECHNICAL FIELD

The present disclosure relates generally to antimicrobial compositions and methods of disinfecting, and more particularly to antimicrobial compositions containing alexidine to disinfect surgical devices, surgical sites of patients and a method to manufacture surgical devices with self-disinfecting properties.

### BACKGROUND

Pre-surgical preparation of the skin with a topical antimicrobial agent is necessary to reduce the likelihood that the patient will contract a hospital-acquired infection during a surgical procedure or post-surgical intervention and maintenance. Typically, the healthcare practitioners, for example, prep nurses, apply a topical antimicrobial agent to a surgical site before the surgical procedure. Similarly, it is essential that surgical instruments (i.e., retractors, forceps, surgical racks, scalpels, surgical knives, scissors, etc.) that breach the skin be disinfected prior to penetrating the skin at the surgical site or accessing an intravenous system. Healthcare practitioners typically sterilize these instruments using heat, gas or gamma radiation sterilization methods well known in the art. Alternatively, where such sterilizing techniques are not available, they may disinfect these instruments by applying or submerging the instruments in an antimicrobial solution, e.g., alcohol, prior to use. Such treatment reduces the infection rate at the site or within the blood stream by hindering the growth of microorganisms or disinfecting a wound, surgical incision, or needle puncture site.

Potential pathogens usually cannot enter the body if the skin and acid mantle remain intact. However, the integrity of the skin barrier is breached during surgical procedures, potentially exposing the surgical site or wound to microorganisms and increasing a patient's risk of infection. Thus, standard surgical procedures require that the skin at the surgical site be disinfected prior to surgery to reduce the patient's risk of infection. It is also required that post-surgery the wound site is kept infection free. Therefore, there is a need in the art for a topical agent that will both kill the transient and resident microorganisms quickly and provide sustained antimicrobial activity throughout the entire surgery or surgical procedure, and even post-surgery.

Furthermore, surgical devices are often implanted in a patient's body where they remain in place for hours and even several days post-surgery. The implantation of these surgical devices, however, poses an increased risk of infection to the patients. As a result, there is an overwhelming concern that these surgical devices are not only disinfected prior to surgery but are able to provide a sustained antimicrobial activity during surgery and after implantation. Therefore, surgical devices that have antimicrobial and self-disinfecting features are useful to prevent post-surgical infections, which can also aid in reducing inflammatory response and faster healing.

Many compositions have been developed for disinfecting surgical devices and surgical sites. For example, chlorhexidine is commonly used as an antimicrobial agent in many disinfecting solutions including topical skin disinfectants, wound closure systems and wound care products. Although chlorhexidine has been useful to some extent in disinfecting solutions for these applications, there are some serious drawbacks to chlorhexidine. For example, it is known that chlorhexidine has the ability to function as a sensitizing agent, and in rare cases it can trigger immediate hypersensitivity in the form of acute anaphylaxis. Another drawback is that chlorhexidine must be present in higher concentrations in order to function as a wide spectrum disinfectant. Higher concentrations of chlorhexidine may cause skin irritation or allergic reactions in some patients. Additionally, chlorhexidine may not be as effective against some microorganisms and/or may not kill microorganisms quickly. Therefore, there is an unmet need for an improved disinfecting solution having a higher level of antimicrobial activity and lower toxicity to the patient's tissue.

Alexidine is a disinfectant that is widely used as an antimicrobial in rinse solutions for oral and ophthalmic (for example, for contact lens cleaning and disinfecting) applications, and has been commercialized in various products, typically at levels of about 100 ppm or less for use with soft contact lenses. As an oral disinfectant, the typical concentration of alexidine is about 1%. Generally, it is desirable to provide the lowest possible level of antimicrobial that is consistent with reliable disinfection in order to provide a generous margin for safety and comfort. To date, alexidine, has not been used as an antimicrobial agent to disinfect surgical devices or surgical sites, or to reduce site infection post-surgery.

Both alexidine and chlorhexidine are antimicrobial agents known as bis-biguanides. Both antimicrobial agents possess the biguanide and the hexamethylene structures. Alexidine however, differs from chlorhexidine by possessing ethyl-hexyl end groups instead of chlorophenyl end groups. Due to this structural difference, alexidine is shown to produce lipid phase separation and domains in the cytoplasmic membrane of microbes. The domain formation in the microbial membrane allows alexidine to cause significantly faster alteration in membrane permeability leading to more rapid bactericidal effect as compared to chlorhexidine. The rapid microbial action of alexidine makes it especially beneficial in a skin disinfectant composition which may be utilized in situations requiring quick disinfection (i.e., skin preparation prior to an emergency trauma surgery), as well as in providing long term antimicrobial protection post-surgery. Alexidine is also shown to promote apoptosis as an anti-cancer agent and possess anti-inflammatory and anti-diabetic properties which can aid in rapid wound healing. Furthermore, Alexidine is also shown to have significantly lower risk of causing IgE (Immunoglobulin E) mediated hypersensitivity as compared to chlorhexidine.

Accordingly, the antimicrobial compositions and methods disclosed herein are directed at overcoming one or more of the disadvantages in currently available antimicrobial compositions for surgical sites and surgical devices by using alexidine.

### SUMMARY

In accordance with one aspect of the disclosure, an antimicrobial composition used to disinfect a surgical device or a surgical site is disclosed. The antimicrobial composition includes alexidine, a solvent, an optional carrier polymer and one or more excipients or additives. The antimicrobial composition may be used as a surface application on a surgical instrument or a surgical device when applicable.

In accordance with another aspect of the disclosure, a method of disinfecting a surgical device is disclosed. The method includes applying an antimicrobial composition to at least a portion of the surgical device and drying the surgical device. The antimicrobial composition includes alexidine, a solvent, an optional carrier polymer, and one or more excipients or additives.

In accordance with another aspect of the disclosure, a method of coating a surgical device to provide it with antimicrobial properties is disclosed. The method includes applying an antimicrobial composition with alexidine to at least a portion of the surgical device and drying the surgical device. The antimicrobial composition includes alexidine, a solvent, an optional carrier polymer, and one or more excipients or additives.

### BRIEF DESCRIPTION OF THE FIGURES

FIG. 1 shows photographic images of the zone of inhibition results on day 1 obtained in the zone of inhibition assay using Staphylococcus aureus for the antimicrobial sutures in Example 4 according an aspect of the disclosure.

FIG. 2 shows photographic images of the zone of inhibition results on day 7 obtained in the zone of inhibition assay using Staphylococcus aureus for the antimicrobial sutures in Example 4 according an aspect of the disclosure.

### DETAILED DESCRIPTION

Before the present methods and devices are disclosed and described, it is to be understood that the methods and devices are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

As used herein, the term “alexidine” includes alexidine, alexidine base, alexidine hydrochloride, alexidine dihydrochloride, alexidine monoacetate, alexidine diacetate, alexidine gluconate, alexidine digluconate and mixtures thereof. In general, the alexidine used in the antimicrobial composition may be prepared by any of the processes known in the art for manufacturing alexidine.

As used herein, the term or phrase “disinfect” or “disinfecting” may, in one aspect, refer to, without limitation, the destruction and removal of viable microorganisms from a material including the spores of the microorganisms. The terms “disinfect” and “disinfecting” may, also without limitation, refer to a reduction of viable microorganisms and their spores and does not necessarily imply the complete removal of all viable microorganisms and their spores.

As used herein, the term or phrase “antimicrobial agent” may, in one aspect, refer to, without limitation, agent(s) that are responsible for, or cause the destruction and removal of viable microorganisms from a material including the spores of the microorganisms. The antimicrobial agent may, also without limitation, refer to agents that effect a reduction of viable microorganisms and their spores and does not necessarily imply the complete removal of all viable microorganisms and their spores.

As used herein, the term “surgical device” as used herein is intended to broadly mean any implement or instrument used during surgery either to shape, cut or form tissue or bone, or implanted or otherwise remain within tissue or bone. Examples of surgical instruments for use in the present disclosure include various forceps, occluders, dilators, trocars, retractors, hemostats, sutures, tissue clamps, and needle holders. Surgical instruments may also include a drill, reamer, implant, bone plate, scalpel, screws, etc.

As used herein, the term “excipient” refers to a non-therapeutic agent added to the antimicrobial composition for purposes of providing stability to the composition and/or achieving the desired rheological properties. Examples of excipients for use in the present disclosure include binders such as various synthetic polymers, proteins, starches, cellulose, or preservatives.

As used herein, the term “additive” refers to a non-therapeutic agent added to the antimicrobial composition for purposes of providing modified coating properties and/or controlled and extended delivery of a therapeutic agent. Examples of additives for use in the present disclosure include a solvent such as ethyl acetate or an antioxidant such as Irganox® E 201 (Vitamin E).

As used herein, the term “hypoallergenic” refers to a reduced allergic reaction or a reduced tendency to trigger hypersensitivity responses to allergens and may be mediated by IgE (Immunoglobulin E) antibodies.

As used herein, the term “vitamin E” includes alpha, beta, gamma, and delta-tocopherols and their derivatives and conjugates. Vitamin E may include a combination of alpha, beta, gamma, and delta-tocopherols and their derivatives and conjugates.

As used herein, the term “implantable” refers to a surgical device to be positioned partially or wholly at a location within a body, such as within a body vessel. Additionally, the terms “implantation” and “implanted” refer to the positioning of a surgical device at a location, partially or wholly, within a body, such as within a body vessel or muscle.

As used herein, the term or phrase “surgical site” may, in one aspect, refer to, without limitation, the exact location where a surgical procedure is to be performed on a surgical patient. Alternatively, the term “surgical site” may, without limitation, refer to a predetermined location on a surgical patient that is sufficiently near or in close proximity to the exact location of a surgical procedure to be performed.

As used herein, the terms “minimum inhibitory concentration” and “MIC” are used interchangeably and refer to the minimum concentration of an antibacterial agent in a given culture medium below which bacterial growth is not inhibited.

As used herein, the terms “minimum bactericidal concentration” or “MBC” are used interchangeably and refer to the minimum concentration of an antibacterial agent in a given culture medium below which bacterial growth is not eliminated.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present methods and devices may be understood more readily by reference to the following detailed description of preferred embodiments and the Examples included therein and to the Figures and their previous and following description.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

The Antimicrobial Composition

The present disclosure makes use of alexidine in antimicrobial compositions for medical and non-medical applications. For example, the antimicrobial compositions disclosed herein may be used to disinfect surgical devices used in a surgical procedure. The antimicrobial compositions may also be used to provide surgical devices with self-disinfecting properties or to manufacture such devices with these properties. Additionally, the antimicrobial compositions may be used to disinfect a surgical site of a patient prior to surgery or to cleanse and disinfect skin generally.

In certain aspects of the disclosure, the antimicrobial composition may include alexidine, an excipient or an additive, a solvent and an optional carrier polymer. The antimicrobial composition may be in various forms depending on how the antimicrobial composition is used. In one aspect, these forms may include a solution, gel, suspension or solid dispersion.

The antimicrobial composition disclosed herein shows surprising and unexpected broad spectrum activity against various microorganisms. In particular, the antimicrobial effects obtained from antimicrobial compositions of the present disclosure, which include alexidine far exceed the results obtained from comparative antimicrobial compositions, which include chlorhexidine.

In one aspect, the antimicrobial composition has a broad spectrum antimicrobial effect against the gram positive bacteria, gram negative bacteria, and fungal pathogens responsible for infections. For example, the antimicrobial composition is effective against gram positive bacteria such as Staphylococcus aureus, gram negative bacteria such as Pseudomonas aeruginosa or fungi such as Candida albicans to various extents. Therefore, methods of using the antimicrobial composition described herein may be provided for the prevention of infections caused by these microorganisms.

The antimicrobial composition may further include various therapeutic agents. In one aspect, the antimicrobial composition may promote wound healing. Wound healing may be achieved through alexidine alone or the incorporation of other suitable agents into the antimicrobial composition known in the art to promote wound healing. Additionally, the antimicrobial composition may also prevent the formation of a biofilm on the surface of the surgical device.

A surprising and unexpected finding of the antimicrobial composition disclosed herein is that it has been shown to be hypoallergenic, in particular as compared to antimicrobial compositions based on chlorhexidine. In another aspect, the antimicrobial composition may also be less likely to cause adverse reactions such as hypersensitivity and allergy. Methods and devices for the detection of allergic reactions and responses are described in U.S. Patent Application Publication No. 2014/0187892, the contents of which are incorporated herein by reference in their entirety. In certain aspects, the antimicrobial composition may also aid in reducing inflammatory responses such as erythema, phlebitis, and intimal hyperplasia.

Alexidine

The antimicrobial composition may include one or more of alexidine, alexidine base, alexidine hydrochloride, alexidine dihydrochloride, alexidine monoacetate, alexidine diacetate, alexidine gluconate, or alexidine digluconate. In general, the alexidine used in the antimicrobial composition may be prepared by any of the processes known in the art for manufacturing alexidine.

One advantage of the antimicrobial composition of the present disclosure is that a greater antimicrobial effect is achieved using a lower concentration of alexidine than other antimicrobial agents, such as chlorhexidine. In one aspect, the antimicrobial composition may have a concentration ranging from 0.0001 wt % to 4.0 wt % of alexidine. In another aspect, the antimicrobial composition may have a concentration ranging from 0.01 wt % to 2.0 wt % of alexidine. In another aspect, the antimicrobial composition may have a concentration of at least about 0.05 wt % of alexidine. The concentration of alexidine in the antimicrobial composition, however, is not limited in the present disclosure.

In certain aspects of the present disclosure, the antimicrobial composition may not include chlorhexidine, triclosan, or silver. For example, in some aspects alexidine may be the only antimicrobial agent present in the antimicrobial composition.

Excipient, Additive, Solvent

In certain aspects of the disclosure, the excipient used in the disinfecting and antimicrobial composition may include a common excipient or an additive such as sodium citrate, sodium chloride, sodium saccharin, phenoxyethanol, hydroxybenzonates, sulfobetaine, ethylene glycol, etc. Other suitable excipients and additives are also contemplated for use in the present disclosure. For example, in one aspect, the antimicrobial composition may include an antioxidant such as Vitamin E. Irganox® E 201 is an example of an antioxidant manufactured by BASF that may be useful in the antimicrobial composition.

Solvent used in the antimicrobial composition may include water, an organic solvent, or any combination thereof. Suitable organic solvents, for example, may include without limitation, alcohol, dimethyl formamide, tetrahydrofuran (THF), ethyl acetate, butyl acetate, acetone, methyl ethyl ketone (MEK), citric acid, or mixtures thereof. Other suitable organic solvents may include, without limitation, methanol, butanol, t-butanol, ethylene glycol, diethylene glycol, triethylene glycol, polyethylene glycol, glycerin, and propylene glycol, etc.

In one aspect according to the disclosure, the solvent used in the antimicrobial composition is an alcohol, such as isopropanol, methanol or ethanol or mixtures thereof. More than one solvent may be used in the disinfecting or antimicrobial composition. For example, in certain aspects, the solvent may comprise tetrahydrofuran (THF) and methanol, THF and ethanol, or THF and isopropyl alcohol, or THF and citric acid, or THF and isopropyl alcohol and citric acid.

The Carrier Polymer

In one aspect of the disclosure, the antimicrobial composition includes a carrier polymer. The carrier polymer, however, is used more specifically as a part of antimicrobial coating solutions for surgical devices such as sutures. The carrier polymer generally includes a polymer that is soluble in the solvent and also soluble in alexidine. The carrier polymer may also be a biocompatible polymer that does not have any detrimental effect on the disinfecting and antimicrobial properties of alexidine. Furthermore, the carrier polymer may be a polymer that does not adversely affect the integrity of the surgical device in any manner. Suitable carrier polymers include without limitation, polyurethane, polypropylene, polyester, cellulose, poly(methyl methacrylate), acrylate, or combinations, thereof. In one aspect of the present disclosure, the carrier polymer is polyurethane.

Surgical Devices

The disinfecting and antimicrobial composition may be used to disinfect a variety of surgical devices known in the art. The surgical devices may be any implement or instrument used during surgery either to shape, cut or form tissue or bone, or implanted or otherwise remain within tissue or bone. Examples of surgical devices for use in the present disclosure include various retractors, hemostats, tissue clamps, and needle holders. Surgical devices may also include a drill, reamer, implant, bone plate, scalpel, screws, sutures, etc.

Surgical devices contemplated by the present disclosure may also include any endoscopic surgical instruments including, but not limited to, laparoscopic or arthroscopic instruments. The surgical device may be any tool routinely used in endoscopic surgery, including, for example, tissue forceps, hemostats, retractors, clamps, scissors, needle holders and drivers, and cautery tools.

In certain aspects of the disclosure, the surgical device may include without limitation a wound closure system, including sutures, staples, ligation systems and other similar devices; surgical instruments including sutures, hooks, grasper, retractor, positioner, clamp, holder, claspers and other similar instruments; catheters and tubes such as a peritoneal catheter, hydrocephalus shunt catheter, chest drainage tube, and similar devices. The surgical device may also in certain aspects be an instrument such as a surgical retractor, forceps, surgical racks, bone hooks, scalpels, surgical knives, scissors, tracheal dilator, tracheal tubes, surgical probes, speculums, surgical depressors and dilators, syringes, spatulas, endoscopes, gloves or arthroscopes. Other surgical devices, however, are contemplated and the present disclosure is not limited in this regard.

The surgical device of some aspects may be, without limitation, a surgical screw of any variety, a spinal or other orthopedic plate, a surgical rod, an interbody spinal device, a vertebral disc arthroplasty device, a nucleus replacement device, a corpectomy device, a vertebrectomy device, a mesh device, a facet fixation or arthroplasty device, a structural bone graft, a staple, a tether of synthetic material or wire, or other spinal fixation instrumentation, an intramedullary nail, an external fixation device, a hip prosthesis or therapeutic device, a knee prosthesis or therapeutic device, or an instrument useful with any of the previously recited devices.

The surgical devices may also include neuromodulators including deep brain stimulators (DBS), various pain control devices, and lead systems for stimulation of the spinal cord, muscles, and other nerves of the body; implantable diagnostic devices for monitoring cardiac function; cochlear implants; and drug pumps for administering periodic or demand based pharmacological therapy. Surgical devices may also include gastric band systems, vascular access ports, injection ports, implantable cardioverter defibrillators, heart pacemaker, intra-uterine device, coronary stent, and tympanostomy tubes.

In certain aspects, the antimicrobial compositions of the present disclosure may be used to form a surgical device or a component of a surgical device. In one aspect, the antimicrobial composition may include a layer or a coating on a surface of a surgical device that is intended for contact with the body. In another aspect, the antimicrobial composition provides an antimicrobial effect to the surgical device and to the patient during surgery and after surgery.

Methods of Disinfecting Surgical Devices

In certain aspects of the present disclosure, a method of disinfecting a surgical device is provided. The method of disinfecting may include applying the antimicrobial composition to at least a portion of the surgical device and then drying the surgical device. In one aspect, the surgical device may be soaked in the antimicrobial composition for a period of time of about 5 seconds to about 5 minutes. In another aspect, the surgical device may be soaked in the disinfecting composition for a period of time of about 2 seconds to about 2 minutes. In certain aspects, the surgical device is soaked in the antimicrobial composition for at least 4 seconds. However, the surgical device may be soaked in the disinfecting composition for longer periods of time without adversely affecting the integrity of the surgical device. One advantage of the present disclosure is that the antimicrobial composition is a rapid disinfectant and therefore, does not require long periods of time to effectively disinfect the surgical device. This advantage is particularly valuable during surgical procedures where it is necessary to immediately facilitate sterilization and/or disinfection of the surgical device.

In certain aspects of the present disclosure, the surgical device may be dried at room temperature such that the solvent evaporates. In one aspect, the surgical device may be dried by removing the solvent from the antimicrobial composition. In another aspect, the solvent may be removed from the antimicrobial composition and an amount of alexidine may remain on a surface of the surgical device. The remaining amount of alexidine on the surgical device may provide an antimicrobial effect to the surgical device, which will serve to further prevent infection during the surgical procedure and in some cases, after the surgical procedure.

The alexidine may remain on the surface of the surgical device in its free form. Alternatively, the alexidine may become embedded in the matrix of the carrier polymer, which may provide a longer term antimicrobial effect for the patient through the surgical device. In certain aspects of the disclosure, the antimicrobial composition may be infused, absorbed, penetrated, coated, adhered into or onto a surface of the surgical device.

The antimicrobial composition may be used to form an antimicrobial coating on the surgical device. The antimicrobial composition may be applied to the surgical device using any means known to those skilled in the art. For example, the surgical device may be soaked in the antimicrobial composition for a specified time period until a coating is formed. In one aspect of the present disclosure, the antimicrobial composition may be sprayed onto any of the surfaces of the surgical device. In other aspects, the surgical device may be dip coated in the antimicrobial composition. Alternatively, the antimicrobial composition may be brush coated, die coated, wiped, painted or rolled onto the surfaces of the surgical device. In yet other aspects, extrusion methods may be useful to form either an antimicrobial layer on the surgical device or for bulk distribution of alexidine in the device. Any of these techniques or methods of applying the antimicrobial composition may be used in combination and/or repeated multiple times to form the desired antimicrobial coating.

In one aspect, the antimicrobial composition may be a composition comprising an antimicrobial agent, an excipient or an additive, and a polymer, wherein the antimicrobial agent is alexidine and the antimicrobial composition is used to coat sutures including silk sutures, nylon sutures, polypropylene sutures, polyester sutures, polyglycolide or polyglycolic acid (PGA) sutures, poly(glycolide-co-(εcaprolactone)) (PGCL) sutures, rapid polyglycolic acid (RPGA) sutures, and polydioxanone (PDA) sutures.

Surgical Sites

In certain aspects of the present disclosure, the antimicrobial composition disclosed herein may be used to disinfect the surgical site of a patient. The surgical site of the patient may include the outer skin surface, an open wound or a body cavity. The surgical site may also include any internal tissue of the body. For example, muscle tissue, connective tissue, epithelial tissue and nervous tissue are all contemplated as being part of the surgical site. In some aspects, the antimicrobial composition may be used to disinfect a urinary bladder, genitourinary apparatus, intestine, peritoneal cavity, abdominal cavity, or similar space.

In one aspect, the antimicrobial composition may become infused, absorbed, penetrated, coated, and adhered into or onto the surgical site of the patient. In another aspect, the antimicrobial composition forms a film on the surface of the surgical site. For example, the antimicrobial composition may form an antimicrobial film on the surface of the patient's skin. This antimicrobial film may provide an additional safeguard against infection for the patient.

Methods of Disinfecting Surgical Sites

In certain aspects of the present disclosure, a method of disinfecting the surgical site of a patient is provided. The method of disinfecting the surgical site may include applying the antimicrobial composition to the surgical site and then drying the surgical site. In one aspect, the antimicrobial composition may be applied directly to the skin surface to disinfect the surgical site. Alternatively, an applicator may be used to topically apply the antimicrobial composition to the surgical site. Suitable applicators may include pre-soaked towels, sponges, swab sticks or cloths. These applicators may be composed of cotton, polytetrafluoroethylene (PTFE), cellulose, polyethelene, or polypropylene.

Application of the antimicrobial composition may occur either pre-operatively or post-operatively. After application, the surgical site may be air dried, evaporating the antimicrobial composition. In one aspect, an amount of alexidine remains on the surface of the surgical site and provides an antimicrobial effect to the surgical site.

In one aspect, a skin cleansing wipe moistened with an antimicrobial solution is disclosed. The antimicrobial solution comprises a solvent and an antimicrobial agent, wherein the antimicrobial agent is alexidine and the wipe is suitable to disinfect skin. The solvents disclosed herein may be used in the antimicrobial solution. In another aspect, the cleansing wipe is pre-moistened with the antimicrobial solution.

In one aspect, the antimicrobial composition may be an anesthetic gel composition comprising an antimicrobial agent and a gel-forming agent, wherein the antimicrobial agent is alexidine and the anaesthetic gel composition is used to introduce a catheter to a patient.

In certain aspects of the present disclosure, the antimicrobial composition may be used to prepare a patient for surgery by subjecting the patient to a bath of a solution of alexidine. The antimicrobial composition disclosed herein may also be a bath or shower additive, a liquid soap or a skin cleanser.

Surgical Scrub

In certain aspects of the present disclosure, the antimicrobial composition may be useful as a surgical scrub. The surgical scrub may be routinely used by surgeons, nurses and other hospital staff to scrub their hands and forearms prior to surgery. In one aspect, the surgical scrub may be used by a surgeon prior to gloving and gowning. The surgical scrub may be in form of a liquid or foam soap. For example, surgical scrubs that are liquid or foam soaps may be used in conjunction with water to cleanse and disinfect the skin.

Dry sponges and scrub brushes may be used as tools to apply the surgical scrub and mechanically scrub the skin. Alternatively, in certain aspects of the present disclosure, the surgical scrub may be incorporated in the scrub brushes and sponges for convenience. For example, a sponge may be pre-soaked with the surgical scrub and a brush may pre-loaded with the surgical scrub. These pre-soaked or pre-loaded sponges or brushes may be pre-packaged in a sterile wrapper and may be disposable after use. The user of the pre-soaked or pre-loaded sponge or brush may use the brush or sponge to apply the surgical scrub directly to the skin. In some aspects, water may necessary to use the impregnated or pre-loaded sponges and brushes.

Typically, the antimicrobial composition used in a surgical scrub includes alexidine, suitable surfactants and solvents. The surfactant may be any surfactant known to be used in surgical scrubs. In one aspect, the surgical scrub may include more than one surfactant. Suitable surfactants should be compatible with alexidine and may include without limitation, polyethyleneglycol (PEG) esters of fatty acids, PEG ethers of lanolin and fatty acid amides. In another aspect, polyoxvethylenepolyoxypropylene block copolymers may be useful as a surfactant in the surgical scrub. Any of the carrier polymers disclosed herein may also be suitable for use in the surgical scrub.

In certain aspects, the solvent in the surgical scrub may be an alcohol such as isopropanol, or ethanol. Other solvents such as water or dimethylsulfoxide may also be used. In one aspect, the surgical scrub may include more than one solvent. The solvents previously disclosed herein for use in the antimicrobial composition are also suitable for use in the surgical scrub. Any alcohol used in the surgical scrub is typically present in lower concentrations to avoid skin dehydration. In one aspect, the surgical scrub of the present disclosure may be an effective disinfectant yet mild enough on the skin so that it may be used in ample amounts and repeatedly.

The surgical scrub may also include a foaming agent. Typical foaming agents may include amine foaming agents such as cetyldimethylamine oxide, lauryldimethylamine oxide, cetylmethylmyristylamine oxide and dimethylmyristylamine oxide. Other suitable foaming agents known in the art, however, may be used.

The surgical scrub may also include a moisturizer or an emollient to hydrate the skin. Suitable emollients may include without limitation, cetyl lactate, cyclomethicone, dimethicone, and oils. Additionally, the surgical scrub may include additives such as thickeners, emollients, fragrances, perfumes, coloring agents, and preservatives.

Abbreviations

The abbreviations used in the examples are as follows:

| | |
|---|---|
| MBC | Minimum Bactericidal Concentration |
| MIC | Minimum Inhibitory Concentration |
| PGA | Polyglycolic acid |
| PGCL | Poly(glycolide-co-(εcaprolactone)) |
| THF | Tetrahydrofuran |
| TNTC | Number of microbial colonies were Too Numerous To Count |

### EXAMPLES

Although the examples of the present invention will be set forth below, it will become apparent to anyone skilled in the art that the present invention is not limited by them and that various alterations and modifications may be made within the scope of the appended claims.

### Example 1—Composition of Antimicrobial Composition Containing Chlorhexidine

An antimicrobial composition was prepared for application on a surgical device such as peritoneal catheter having the formulation shown in Table A.

TABLE A

| Ingredients | Amount (%) |
|---|---|
| Chlorhexidine | 2 |
| Water | 88 |
| Ethylene glycol | 10 |

### Example 2—Composition of Antimicrobial Composition Containing Alexidine

An antimicrobial composition was prepared for application on a surgical device such as a peritoneal catheter having the formulation shown in Table B.

TABLE B

| Ingredients | Amount (%) |
|---|---|
| Alexidine | 0.5 |
| Water | 89.5 |
| Ethylene glycol | 10 |

### Example 3—Composition to Make Antimicrobial Suture. Dressing. Drainage Tube and Similar Devices

The representative antimicrobial sutures prepared with using three different types of base materials: PGA, polyester and PGCL. Each suture was then dip coated in a bath of the antimicrobial formulation shown in Table C for 30 seconds.

TABLE C

| Ingredients | Amount (%) |
|---|---|
| Alexidine | 2 |
| Methanol | 11.5 |
| THF | 80 |
| Polyether Urethane | 5.5 |
| Other (e.g. excipient and/or additive) | 2 |

After the treatment with the above formulation, the sutures were dried and the alexidine content on the PGA, polyester and PGCL sutures was measured as 44.7 μg/cm, 51.8 μg/cm and 58.9 μg/cm respectively.

### Example 4—Zone of Inhibition Testing of Antimicrobial Sutures

Half-centimeter segments from the antimicrobial PGA, polyester and PGCL sutures were applied on Müller-Hinton agar pre-swabbed with Staphylococcus aureus, and the agar plates were incubated at 37° C. Segments were transferred on fresh agar plates daily for 7 days.

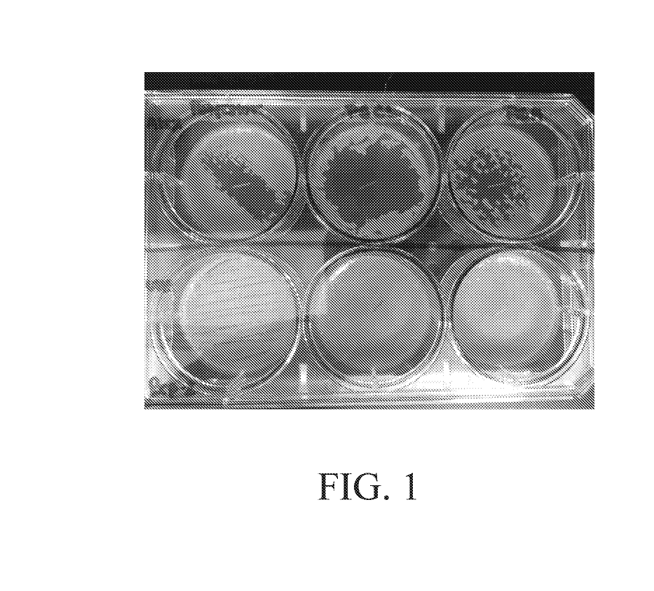

The zones of inhibition on day 1 are shown in FIG. 1. The agar plates in the upper row in FIG. 1 show the zones of inhibition for the alexidine treated sutures. The first, second and third agar plates show the zones of inhibition for the alexidine treated polyester suture, the alexidine treated PGCL suture and the alexidine treated PGA suture. The agar plates in the lower row in FIG. 1 show the zones of inhibition for the untreated sutures. The first, second and third agar plates show the zones of inhibition for the untreated polyester suture, the untreated PGCL suture and the untreated PGA suture. As shown, all three of the alexidine treated sutures (polyester, PGCL, PGA) demonstrate excellent antimicrobial efficacy on day 1.

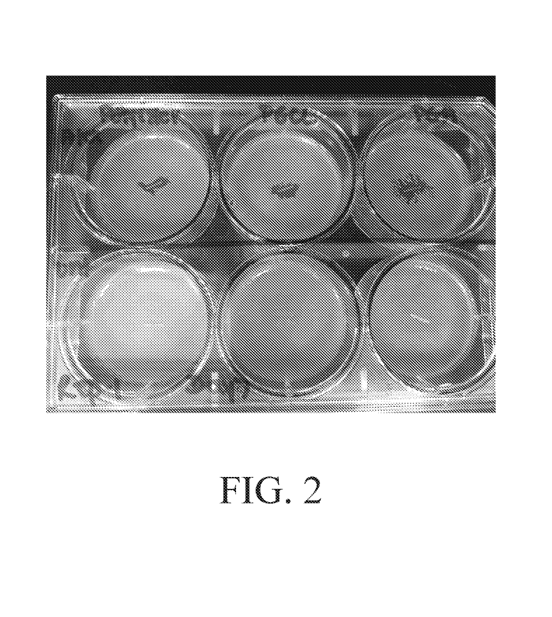

The zones of inhibition on day 7 are shown in FIG. 2. The agar plates in the upper row in FIG. 2 show the zones of inhibition for the alexidine treated sutures. The first, second and third agar plates in the upper row of FIG. 2 show the zones of inhibition for the alexidine treated polyester suture, the alexidine treated PGCL suture and the alexidine treated PGA suture. The agar plates in the lower row in FIG. 2 show the zones of inhibition for the untreated sutures. The first, second and third agar plates show the zones of inhibition for the untreated polyester suture, the untreated PGCL suture and the untreated PGA suture. As shown, all three of the alexidine treated sutures (polyester, PGCL, PGA) demonstrate excellent antimicrobial efficacy on day 7.

### Example 5—Minimum Inhibitory Concentration (MIC) and the Minimum Bactericidal Concentration (MBC) of Alexidine and Chlorhexidine

Description of the Test Method Used:

From the stock solutions of the drugs Alexidine and Chlorhexidine, dilution series was prepared in the wells of a 96-well plate by performing 1:1 dilutions to cover a concentration range of 0-512 ppm. Ten microliters from each of the drug concentration was mixed with 190 μL of culture broth containing approximately 10 CFU/mL of bacteria or yeast species. The test plate was incubated for 18-24 hours after which absorbance of each well was read at 670 nm. The MIC value was the lowest concentration of the drug at which microbial growth was completely inhibited (with the absorbance reading at or below the reading of the drug control wells without any organisms). The wells containing growth should have had higher absorbance reading when compared to the drug control wells. After reading the absorbance for the MIC, 10 μl of each test well was plated onto the surface of Dey Engley Neutralizing Agar (D/E agar) in 6 or 12 well microtiter plates to determine the MBC. The plates were incubated inverted at 37° C. for 24-48 hours after which numbers of colonies were counted. The MBC value was the lowest concentration of the drug at which no growth was observed.

Test Results

The MIC and MBC results for Alexidine as compared to Chlorhexidine are shown in Tables D and E below. Both the MIC and MBC values for Alexidine were lower or similar to that of Chlorhexidine for most microorganisms tested indicating Alexidine as a much potent antimicrobial agent than Chlorhexidine.

TABLE D
MIC of Alexidine versus Chlorhexidine

| | | MIC |
|---|---|---|
| | MIC Alexidine | Chlorhexidine |
| Organism | (μg/mL) | (μg/mL) |
| Staphylococcus aureus | 0.5 | 0.5 |
| Candida albicans | 1 | 2 |
| Pseudomonas aeruginosa | 8 | 8 |
| Enterococcus faecalis | 0.5 | 2 |
| Acinetobacter baumannii | 0.5 | 16 |
| Enterobacter cloacae | 2 | 2 |
| Proteus mirabilis | 1 | 8 |

TABLE E
MBC of Alexidine versus Chlorhexidine

| | MBC | MBC |
|---|---|---|
| | Alexidine | Chlorhexidine |
| Organism | (μg/mL) | (μg/mL) |
| Staphylococcus aureus | 1 | 16 |
| Candida albicans | 1 | 4 |
| Pseudomonas aeruginosa | 128 | 64 |
| Enterococcus faecalis | 2 | 64 |
| Acinetobacter baumannii | 1 | 32 |
| Enterobacter cloacae | 2 | 32 |
| Proteus mirabilis | 2 | 8 |

### Example 6—Comparison of the Kill Time of Alexidine and Chlorhexidine

Description of the Test Method Used:

Alexidine and Chlorhexidine, both at a concentration of 128 ppm were exposed to a Gram positive bacteria (Staphylococcus aureus), a Gram negative bacteria (Pseudomonas aeruginosa), and a fungus (Candida albicans). The challenge concentration for each organism was 10⁴-10⁵ CFU/mL, and the exposure time varied from 0.5-60 minutes. Table F below shows the Time to Kill results for both Alexidine and Chlorhexidine. Complete kill of all three organisms was observed within 0.5-1 minute of Alexidine exposure. In contrast, with Chlorhexidine it took 60 minutes before complete kill was observed for C. albicans and S. aureus, and 5 minutes for P. aeruginosa.

Test Results:

Safety Assessment:

The biocompatibility and toxicity of the antimicrobial compositions of Example 3 were assessed using the six tests described below. The test results show no adverse effects and demonstrate the safety and biocompatibility of surgical devices treated with alexidine. These results surprisingly further show that the antimicrobial composition is hypoallergenic.

### Example 7

The Intracutaneous Injection Test (ISO) was performed. Test rabbits received an intracutaneous injection of the antimicrobial composition of Example 3. All test rabbits increased in body weight and showed no signs of toxicity at the 24 hour, 48 hour and 72 hour observation points.

### Example 8

The Kligman Maximization Test (ISO) was performed. The skin of guinea pigs was treated with the test article extract and exhibited no reaction to the challenge (0% sensitization).

### Example 9

A 28 day Systemic Toxicity via Intramuscular Implantation was performed. The test articles did not demonstrated any local or systemic signs of toxicity when test articles composed of the antimicrobial composition of Example 3 was implanted into the muscle tissue of five rats for 28 days.

### Example 10

The Intramuscular Implantation Test (ISO) was performed. Macroscopic evaluation of the test article implantation site indicated no significant signs of inflammation, encapsulation, hemorrhage, or necrosis. However, microscopic evaluation (histology) of these sites indicated moderate reactivity when compared to the control sites having no implantation.

### Example 11

Intravascular implantation in a Sheep Model to determine safety and efficacy was performed. The test device composed of the antimicrobial composition disclosed in Example 3 was well tolerated. All test animals remained healthy for the entire 7 and 30 day study duration and no signs of organ toxicity were observed. Alexidine-treated device was highly effective in reducing colonization by Staphylococcus aureus (the challenge organism used to infect the implantation site) on the device and the vein tissue surrounding the device. As compared to the un-treated control device, Alexidine-treated device led to 7 to 8 Log₁₀ reduction in bacterial colonization on the device and the surrounding tissue. Alexidine-treated device also led to 99% reduction in weight and 92% reduction in length of the device-associated thrombus when compared to the un-treated control device. There was also significant reduction in inflammatory response from the alexidine treated device compared to the untreated device.

### Example 12

The hemolytic index (HI) of the antimicrobial composition of Example 3 was also tested. The HI of the antimicrobial composition of Example 3 was shown to be comparable to chlorhexidine.

1-50. (canceled)

51. An antimicrobial composition comprising alexidine, a solvent, an optional carrier polymer, and one or more excipients or additives, wherein the antimicrobial composition is used either to disinfect a surgical device or a surgical site or to provide antimicrobial properties to a surgical device during surgery and after surgery.

52. The antimicrobial composition of claim 51, wherein the solvent is selected from the group consisting of: water, an alcohol, dimethyl formamide, tetrahydrofuran, ethyl acetate, butyl acetate, acetone, methyl ethyl ketone (MEK) or mixtures thereof.

53. The antimicrobial composition of claim 52, wherein the alcohol is isopropanol, ethanol, or methanol.

54. The antimicrobial composition of claim 51, wherein the excipient or the additive is selected from the groups consisting of: sodium citrate, sodium chloride, sodium saccharin, phenoxyethanol, hydroxybenzonates, sulfobetaine, and ethylene glycol.

55. The antimicrobial composition of claim 51, wherein the excipient or the additive comprises an antioxidant.

56. The antimicrobial composition of claim 51, wherein the antioxidant comprises vitamin E.

57. The antimicrobial composition of claim 51, wherein the composition has a concentration ranging from 0.0001 wt % to 4.0 wt % of alexidine.

58. The antimicrobial composition of claim 51, wherein the carrier polymer comprises at least one of polyurethane, polypropylene, polyester, cellulose, poly(methyl methacrylate), acrylate, or mixtures thereof.

59. The antimicrobial composition of claim 51, wherein the surgical device is selected from the group consisting of: wound closure system including sutures, staples, ligation systems and other similar devices; surgical instruments including hooks, grasper, retractor, positioner, clamp, holder, claspers and other similar instruments; catheters and tubes such as a peritoneal catheter, hydrocephalus shunt catheter, chest drainage tube, and similar devices.

60. The antimicrobial composition of claim 51, wherein the antimicrobial composition has a broad spectrum antimicrobial effect against the gram positive bacteria, gram negative bacteria, and fungal pathogens responsible for surgical site infections.

61. The antimicrobial composition of claim 51, wherein the antimicrobial composition has an antimicrobial effect on Staphylococcus species such Staphylococcus aureus and Staphylococcus epidermidis, Candida species, Pseudomonas aeruginosa, Enterococcus species, Klebsiella species such as Klebsiella pneumoniae, Providencia stuartii, Proteus mirabilis, Enterobacter species, Acinetobacter species, and Escherichia coli.

62. The antimicrobial composition of claim 51, wherein the antimicrobial composition has a greater antimicrobial effect than a comparative antimicrobial composition comprising chlorhexidine as the antimicrobial agent.

63. A method of disinfecting a surgical device comprising:
applying an antimicrobial composition to at least a portion of the surgical device; and
drying the surgical device, wherein the antimicrobial composition comprises alexidine, a solvent, an optional carrier polymer and one or more excipients or additives.
in the free form or embedded in the matrix of the carrier polymer to provide long term antimicrobial effect from the surgical device.

64. A method of disinfecting a surgical site of a patient, the method comprising:
applying an antimicrobial composition to the surgical site of the patient; and
drying the surgical site of the patient, wherein the antimicrobial composition comprises alexidine and a solvent.

65. A method of forming an antimicrobial coating on a surgical device comprising: applying an antimicrobial composition to a surface of the surgical device, wherein the antimicrobial composition comprises alexidine, a solvent, an optional carrier polymer, and one or more excipients or additives.
